(19) United States
(12) Patent Application Publication
(10) Pub. No.: US 2007/0190078 A1
(43) Pub. Date: Aug. 16, 2007

(54) Filamentous haemagglutinin in the treatment and/or prophylaxis of immune-mediated disorders

(76) Inventors: Kingston Henry Gardon Mills, Rathgar (IE); Peter Mcguirk, Castaheany (IE); Brian Keogh, Clondalkin (IE)

Correspondence Address: HELLER EHRMAN LLP, 275 MIDDLEFIELD ROAD, MENLO PARK, CA, 94025-3506, US

(21) Appl. No.: 10/575,291

(22) PCT Filed: Oct. 14, 2004
(86) PCT No.: PCT/IE04/00139

§ 371 Date: Mar. 2, 2007

(30) Foreign Application Priority Data
Oct. 14, 2003 (IE) 2003/0761

(51) Int. Cl. A61K 39/10; A61K 38/48

(52) U.S. Cl. 424/253.1; 424/94.64

(57) ABSTRACT

Filamentous haemagglutinin (FHA) or a derivative or mutant or fragment or variant or peptide thereof is useful in the prophylaxis and/or treatment of an immune-mediated disorder and/or an autoimmune disease. The FHA may include self or foreign antigens or peptides thereof.

The invention relates to filamentous haemagglutinin (FHA) or a derivative or mutant or fragment or variant or peptide thereof.

Cells of the innate immune system, especially dendritic cells (DC), direct the differentiation of naïve CD4⁺ T cells into functionally distinct Th1, Th2 or regulatory T (Tr) cell subtypes. Activation of immature DC through binding of conserved microbial molecules to pathogen recognition receptors (PRRs), such as Toll-like receptors (TLR) and integrins, is accompanied by maturation and homing to the lymph nodes, where the mature DC presents antigen to the naïve T cells. Activation of DC by pathogen derived molecules plays a critical role in regulating the differentiation of naïve CD4⁺ T cells into distinct T cell subtypes (1, 2). Th1 cells confer protection against intracellular infection but are also associated with inflammatory responses and autoimmune disease, whereas Th2 cells are involved in allergic responses. Tr cells are capable of suppressing Th1 and Th2 responses.

Bordetella pertussis causes a protracted and severe disease, which is often complicated by secondary infection and pneumonia, and can have a lethal outcome in young children. Recovery from infection is associated with the development of B. pertussis-specific Th1 cells and these cells play a critical role in clearance of the bacteria from the respiratory tract However, antigen-specific Th1 responses in the lung and local lymph nodes, are severely suppressed during the acute phase of infection. B. pertussis has evolved a number of strategies to circumvent protective immune responses.

The virulence factor, filamentous haemagglutinin (FHA) from B. pertussis, is capable of inhibiting LPS-driven IL-12 production by macrophages, IL-12 and IFN-γ production in a murine model of septic shock (3) and Th1 responses to an unrelated pathogen, influenza virus, when administered simultaneously to the respiratory tract (4). FHA is considered to function primarily as an adhesin, mediating binding of B. pertussis to the β2-integrins (CR3, CD11b/CD18, αMβ2) via binding to leukocyte response integrin (αVβ33, CD61) and the integrin-associated protein (CD47) complex (5). FHA may also contribute to suppressed Th1 responses during acute infection with B. pertussis by the induction of T cells with regulatory activity, as a result of its interaction with cells of the innate immune system. FHA interacts directly with DC to induce IL-10 and inhibit LPS-induced IL-12 and inflammatory chemokine production (6). The DC generated following interaction with FHA selectively stimulates the induction of Tr1 cells from naïve T cells. Tr1 clones specific for FHA and pertactin (PRN) from B. pertussis were generated from the lungs of acutely infected mice. These Tr1 cells secreted high levels of IL-10 and inhibited protective Th1 responses against B. pertussis in vitro and in vivo (6). These findings demonstrated a novel function for Tr1 cells, exploited by a respiratory pathogen to evade protective immunity, and provided evidence that these regulatory cells are induced by DC in which IL-10 production is activated and IL-12 suppressed following interaction with a pathogen-derived molecule.

Multiple sclerosis (MS) is an autoimmune disease that affects the central nervous system. Individuals with this disease have autoreactive T cells (T cells that recognize self antigens), which together with interleukin (IL)-1β and tumour necrosis factor (TNF)α, participate in the formation of inflammatory lesions along the myelin sheath of nerve fibres. The cerebrospinal fluid (CSF) of patients with MS contains activated T cells, which infiltrate the brain tissue and cause the characteristic inflammatory lesions, destroying the myelin. Experimental autoimmune encephalomyelitis (EAE) is an animal model for MS. It is induced in mice or rats by injection of mylein basic protein (MBP) or myelin oligodendrocyte glycoprotein (MOG) or peptides thereof with complete Freund's adjuvant. The disease can also be induced by transfer of MBP or MOG-specific T cells that secrete IFN-γ (called Th1 cells). The animals develop cellular infiltration of the myelin sheaths of the central nervous system, resulting in demyelination and eventually paralysis. The clinical signs and pathological changes resemble MS.

Crohn's disease and ulcerative colitis are inflammatory bowel diseases in humans. These autoimmune diseases are inflammatory conditions of the intestine mediated by CD4⁺ T cells. Regulatory T cells (Tr cells) prevent the development of autoimmune diseases in normal individuals. Injection of CD45RB^{high} (naïve) T cells can induce colitis in severe combined immunodeficient (SCID) mice, which can be prevented by co-transfer of CD45RB^{low} or CD4⁺ CD25⁺ regulatory T cells (7). Furthermore elimination of CD45RB^{low} or CD4⁺ CD25⁺ regulatory T cells leads to spontaneous development of various autoimmune diseases in otherwise normal mice or rats (8).

A method of inducing anti-inflammatory cytokines by cells of the innate immune system or for modulating innate immune cells to direct the induction of Tr cells in vivo would have valuable potential for the treatment of inflammatory and autoimmune diseases and allergy.

Statements of Invention

According to the invention there is provided a method for the prophylaxis and/or treatment of an immune-mediated disorder comprising the step of administering an agent comprising filamentous haemagglutinin (FHA) or a derivative or mutant or fragment or variant or peptide thereof.

The invention also provides a method for the prophylaxis and/or treatment of an autoimmune disease comprising the step of administering an agent comprising filamentous haemagglutinin (FHA) or derivative or mutant or fragment or variant or peptide thereof.

The invention further provides use of an agent comprising filamentous haemagglutinin (FHA) or a derivative or mutant or fragment or variant or peptide thereof for the prophylaxis and/or treatment of an immune-mediated disorder.

The invention also provides use of an agent comprising filamentous haemagglutinin (FHA) or derivative or mutant or fragment or variant or peptide thereof for the prophylaxis and/or treatment of an autoimmune disease.

In one embodiment the filamentous haemagglutinin (FHA) is derived from Bordetella pertussis or Bordetella bronchisepetica or Bordetella parapertussis or related molecules from other bacteria. Related molecules may include proteins from other bacterial with sequences homologous to those in FHA.

In one embodiment of the invention the agent comprises FHA or derivative or mutant or fragment or variant or peptide thereof or a product of cells activated by these materials.

In one embodiment the agent comprises FHA in combination with self or foreign antigens or peptides thereof.

In one embodiment of the invention the agent promotes the induction of anti-inflammatory cytokines in vivo.

In one embodiment of the invention the agent promotes the generation of Tr cells in response to a self antigen.

In another embodiment of the invention FHA acts as an immunomodulator in vivo to promote the induction of Tr cells to co-administered self or foreign antigens.

Preferably the self antigen is selected from any one or more of glutamic acid decarboxylase 65 (GAD 65), native DNA, myelin basic protein, myelin proteolipid protein, acetylcholine receptor components, thyroglobulin, thyroid stimulating hormone (TSH) receptor, Japanese cedar pollen antigens, ragweed pollen antigens, rye grass pollen antigens, and dust mite antigens and feline antigens for animal, histocompatibility antigens, antigens involved in graft rejection and an altered peptide ligand. The antigens involved in graft rejection comprise antigenic components of the graft to be transplanted into the heart, lung, liver, pancreas, kidney of graft recipient and neural graft components.

The self antigen may also be selected from any one or more of a myelin protein, beta amyloid protein, amyloid precursor protein and collagen and peptide thereof.

Preferably the myelin protein is myelin basic protein or peptide thereof. The myelin basic protein is myelin oligodendrocyte glycoprotein synthetic peptide, most preferably a MOG peptide (35-55).

In one embodiment of the invention the agent modulates inflammatory cytokine production.

In one embodiment the agent promotes the induction of anti-inflammatory cytokines.

In another embodiment of the invention the immunomodulatory effects of FHA on cells of the innate immune system is enhanced by co-activation with a Toll-like receptor ligand. The Toll-like receptor (TLR) ligand may be LPS or other toll-like receptor ligands, selected from any one or more of CpG motifs, dsRNA, Poly (I:C) and Pam3Cys.

In one embodiment of the invention FHA promotes IL-10 and TGF-β production by macrophages and dendritic cells.

In one embodiment of the invention FHA promotes IL-6 production by macrophages and dendritic cells.

In a further embodiment of the invention FHA synergises with LPS or other TLR ligands to promote IL-10, TGFβ and IL-6 production by macrophages and dendritic cells.

In one embodiment of the invention FHA induces expression of TGFβ mRNA.

In another embodiment FHA inhibits inflammatory cytokines, chemokines or other inflammatory mediators. The inflammatory cytokine may be selected from any one or more of TNF-α, IFN-γ, IL-2, IL-12, IL-1, IL-23 and IL-27.

The inflammatory cytokine may be macrophage inflammatory protein-1α or macrophage inflammatory protein-1β.

In one embodiment FHA promotes dendritic cell maturation into a semi-mature phenotype.

In one embodiment FHA promotes dendritic cell maturation following co-activation with TLR-ligands.

In another embodiment FHA inhibits TLR-ligand-induced dendritic cell activation.

Preferably the FHA is substantially endotoxin free. The FHA may comprise less than 300 pg endotoxin/μg protein.

In one embodiment of the invention the FHA is in the form of an immmunomodulator, adjuvant, immunotherapeutic or anti-inflammatory agent.

In one embodiment of the invention the agent modulates inflammatory cytokine production induced by infection or trauma.

In another embodiment the immune-mediated disorder is sepsis or acute inflammation induced by infection, trauma or injury. The disorder may be multiple sclerosis.

In one embodiment of the invention the immune-mediated disorder is selected from any one or more of multiple sclerosis, Crohn's disease, inflammatory bowel disease, type 1 diabetes, rheumatoid arthritis and psoriasis. Other immune-mediated disorders include any one or more of diabetes mellitus, arthritis (including rheumatoid arthritis, juvenile rheumatoid arthritis, osteoarthritis, psoriatic arthritis), myasthenia gravis, systemic lupus erythematosis, autoimmune thyroiditis, dermatitis (including atopic dermatitis and eczematous dermatitis), Sjogren's Syndrome, including keratoconjunctivitis sicca secondary to Sjogren's Syndrome, alopecia areata, allergic responses due to arthropod bite reactions, aphthous ulcer, iritis, conjunctivitis, keratoconjunctivitis, ulcerative colitis, asthma, allergic asthma, cutaneous lupus erythematosus, scieroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, interstitial lung fibrosis, Alzheimers disease and coeliac disease.

In another embodiment of the invention the immune-mediated disorder is colitis, asthma or atopic disease.

Preferably the agent is in a form for oral, intranasal, intravenous, intradermal, subcutaneous or intramuscular administration. The agent may be administered repeatedly.

The invention further provides an immunomodulator comprising FHA.

The invention also provides a recombinant FHA having immunomodulatory effects.

The invention further provides a vaccine comprising FHA or derivative or mutant or fragment or variant or peptide thereof. The vaccine may comprise FHA or derivative or mutant or fragment or variant or peptide thereof and an antigen.

Preferably the FHA and antigen are present in a by weight ratio range of 0.01:1 to 100:1. Preferably the FHA and antigen are present in a molar ratio of 1:10 to 10:1.

The invention also provides antibodies to FHA or derivative or mutant or fragment or variant or peptide thereof.

The invention also provides a product comprising FHA or derivative or mutant or fragment or variant or peptide thereof in combination with an antigen, where said antigen is selected from a self-antigen and a foreign antigen.

In one embodiment the product comprises FHA or derivative or mutant or fragment or variant or peptide thereof in combination with a TLR ligand.

In another embodiment the product comprises FHA or derivative or mutant or fragment or variant or peptide thereof in combination with a TLR ligand and a self antigen.

Preferably the TLR ligand is a pharmaceutically acceptable TLR ligand. The TLR ligand may be selected from any one or more of CpG motifs, dsRNA, Poly (I:C) and Pam3Cys.

The product may comprise a derivative or mutant or fragment or variant or peptide thereof or a product of cells activated by these materials.

The invention also provides a pharmaceutical composition comprising FHA or derivative or mutant or fragment or variant or peptide thereof.

In one embodiment of the invention the pharmaceutical composition comprises FHA or derivative or mutant or fragment or variant or peptide thereof as adjuvant for immunization with a self or foreign antigen.

In another embodiment the pharmaceutical composition comprises FHA or derivative or mutant or fragment or variant or peptide thereof in combination with an antigen, where said antigen is selected from a self-antigen and a foreign antigen.

The FHA may comprise a derivative or mutant or fragment or variant or peptide thereof or a product of cells activated by these materials.

The self antigen may be selected from any one or more of glutamic acid decarboxylase 65 (GAD 65), native DNA, myelin basic protein, myelin proteolipid protein, acetylcholine receptor components, thyroglobulin, thyroid stimulating hormone (TSH) receptor, Japanese cedar pollen antigens, ragweed pollen antigens, rye grass pollen antigens for pollen, and dust mite antigens and feline antigens for animal, histocompatibility antigens, antigens involved in graft rejection and an altered peptide ligand. The antigens involved in graft rejection may comprise antigenic components of the graft to be transplanted into the heart, lung, liver, pancreas, kidney of graft recipient and neural graft components.

The self antigen is preferably selected from any one or more of a myelin protein, beta amyloid protein, amyloid precursor protein and collagen and peptides thereof. The myelin protein may be myelin basic protein or peptides thereof. The myelin basic protein may be myelin oligodendrocyte glycoprotein synthetic peptide. The myelin basic protein may be a MOG peptide (35-55).

In one embodiment the pharmaceutical composition comprises FHA or derivative or mutant or fragment or variant or peptide thereof in combination with a TLR ligand. Preferably the TLR ligand is a pharmaceutically acceptable TLR ligand.

The invention further provides use of an agent comprising FHA or derivative or mutant or fragment or variant or peptide or product of cells activated by the agent for the treatment and/or prophylaxis of an inflammatory and/or immune-mediated disorder.

The invention also provides use of an agent comprising FHA or derivative or mutant or variant or peptide or product of cells activated by the agent for the prophylaxis and/or treatment of diseases or conditions involving Toll-like receptor dependent signalling.

The invention also provides use of an agent comprising FHA or derivative or mutant or fragment or variant or peptide or product of cells activated by the agent for the prophylaxis and/or treatment of asthma or allergy.

The invention also provides use of an agent comprising FHA or a derivative or mutant or fragment or variant or peptide thereof or product of cells activated by the agent for the prophylaxis of an immune-mediated disorder.

The invention also provides use of an agent comprising FHA or a derivative or mutant or fragment or variant or peptide thereof or product of cells activated by the agent for the prophylaxis and/or treatment of multiple sclerosis.

The invention also provides use of an agent comprising FHA or a derivative or mutant or fragment or variant or peptide thereof or product of cells activated by the agent for the prophylaxis and/or treatment of a disease selected from any one or more of multiple sclerosis, Crohn's disease, inflammatory bowel disease, type 1 diabetes, rheumatoid arthritis and psoriasis.

The invention also provides use of an agent comprising FHA or a derivative or mutant or fragment or variant or peptide thereof or product of cells activated by the agent for the prophylaxis and/or treatment of colitis, inflammatory bowel disease or asthma or allergy.

In a further embodiment, the invention provides methods for purification of FHA, including methods for providing substantially LPS-free FHA. The invention provides a method for preparing a substantially pure preparation of FHA comprising the steps of dialysing a preparation of FHA to denature the protein and expose contaminating endotoxin and removing residual contaminating endotoxin. The endotoxin may be removed using a detergent. In one case the method comprises the steps of:
priming a purification column;
adding the dialysed FHA preparation;
washing with detergent; and
eluting a substantially purified protein.

The term immune-mediated disorder is taken throughout to include any disorder where immune responses contribute to the pathogenesis of the disease, and includes but is not confined to autoimmune diseases.

The term autoimmune disease refers to one of a number of unrelated disorders caused by inflammation and destruction of tissues by the body's own immune system and involves the generation of cellular or humoral immune responses against components or products of its own tissue, treating them as foreign.

The terms derivative or mutant or fragment or variant or peptide as used herein are understood to include any molecule or macromolecule consisting of a functional portion of FHA. Fragments or variants or peptides may be prepared by techniques commonly known to the person skilled in the art. These include peptides or fragments corresponding to the regions of FHA that interact with CD11b/CD18 or CD47/CD61 and may include RGD motifs. Preliminary data with synthetic peptides corresponding to RGD-containing regions of FHA suggest that they may be capable of inducing IL-10 and/or inhibiting IL-12 production from macrophages or dendritic cells.

The term antigen refers to a molecule which can initiate a humoral and/or cellular immune response in a recipient of the antigen. A humoral and/or cellular immune response may include, for example, production of an antibody specific for the antigen, or induction of T cells which recognize or bind to the antigen. The term antigen is taken throughout to include any substance that binds specifically to an antibody or T cell receptor. The term self- or auto-antigen is taken to mean an endogenous antigen on self-tissue or cell in the body, which is not foreign. The term foreign antigen is taken to mean an antigen from a pathogen (bacteria, virus, fungi or parasite).

Antigens involved in autoimmune diseases, allergy, and graft rejection may be used in the compositions and methods of the invention. Examples of antigens involved in autoimmune disease include myelin oligodendrocyte glycoprotein (MOG), glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens. Examples of antigens involved in graft rejection include antigenic components of the graft to be transplanted into the graft recipient such as heart, lung, liver, pancreas, kidney, and neural graft components. An antigen can also be an altered peptide ligand useful in treating an autoimmune disease.

Examples of miscellaneous antigens which can be can be used in the compositions and methods of the invention include, beta amyloid protein and amyloid precursor protein.

The term adjuvant is taken to include a substance used in conjunction with an antigen to enhance the immune response to the antigen in vivo.

The term immunomodulator is taken to include any molecule, including those derived from bacteria, viruses, parasites or fungi pathogens, that modulates, ie increases and/or decreases, the responses of cells of the immune system.

The term semi-mature phenotype of a dendritic cell (DC) is an intermediate phenotype between an immature and a full mature DC. An immature DC is one that resided in a tissue and has not been stimulated. A mature DC is generated from an immature DC following stimulation with a toll-like receptor ligand alone or with cytokines. Maturation means enhancement of expression of co-stimulatory molecules and MHC molecule on the cell surface.

### BRIEF DESCRIPTION OF THE INVENTION

The invention will be more clearly understood from the following description thereof, given by way of example with reference to the accompanying drawings in which:

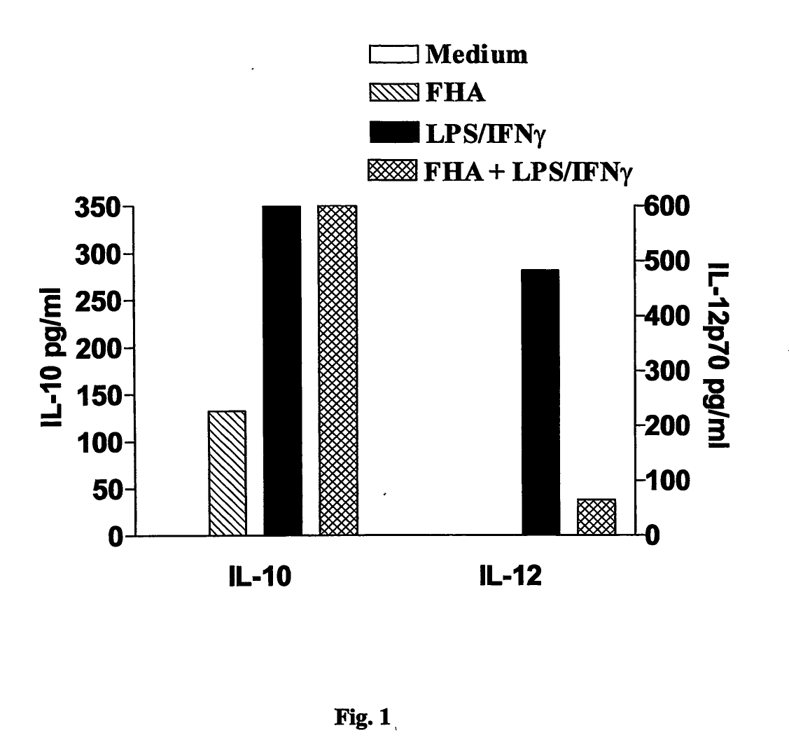

FIG. 1 is a graph showing that FHA stimulates IL-10 and inhibits IL-12 production from human monocytes. CD14⁺ monocytes were purified from human peripheral blood mononuclear cells from a normal donor using positive selection with MACS microbeads and an autoMACS sorting instrument. Monocytes (1×10⁶/ml) were stimulated with medium only, FHA (5 μg/ml) LPS (1 μg/ml) and IFN-γ (20 ng/ml) or FHA, LPS and IFN-γ. Supernatants were removed after 24 hours and IL-10 and IL-12p70 concentrations determined by two site ELISA;

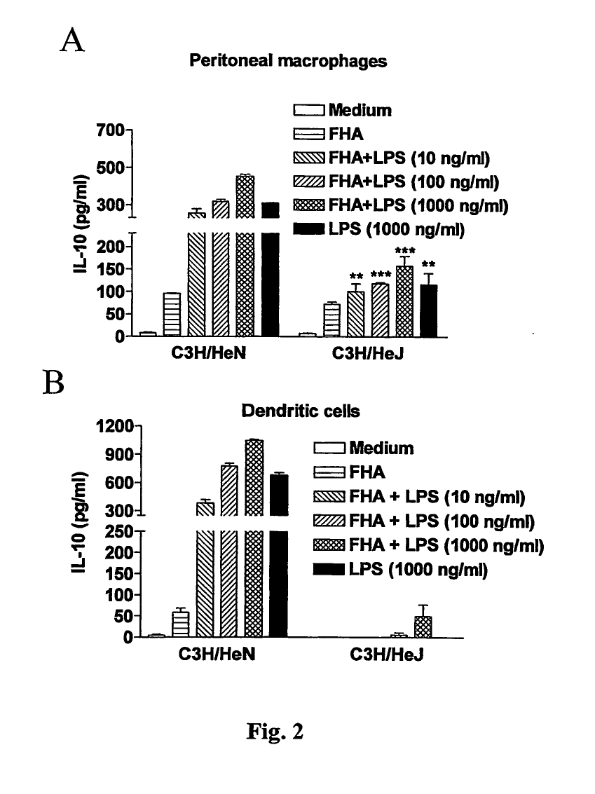

FIG. 2 is a graph showing that FHA stimulates IL-10 production from murine macrophages (A) and dendritic cells (B) and that this effect is augmented by LPS signalling through Toll-like receptor-4 (TLR-4). Macrophages were recovered from the peritoneal cavity of normal C3H/HeN and TLR-4-defective C3H/HeJ mice by peritoneal lavage. Immature bone marrow derived dendritic cells were generated from bone marrow obtained from femurs and tibia of normal C3H/HeN and TLR-4-defective C3H/HeJ mice and cultivated for 7 days with a GM-CSF-containing supernatant. Peritoneal macrophages or dendritic cells (1×10⁶/ml) were stimulated with medium only, FHA (5 μg/ml) LPS (1000 ng/ml), medium only or FHA (5 μg/ml) and LPS (10-1000 ng/ml). Supernatants were removed after 24 hours and IL-10 concentrations determined by two site ELISA;

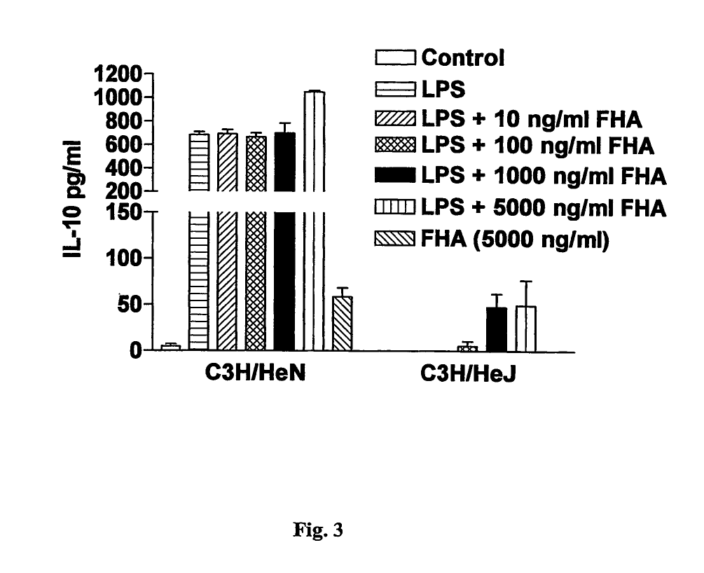

FIG. 3 is a graph showing the effect of increasing concentrations of FHA on LPS-induced IL-10 production. Immature bone marrow derived dendritic cells were generated from bone marrow obtained from femurs of normal C3H/HeN and TLR-4-defective C3H/HeJ mice and cultivated for 7 days with a GM-CSF-containing supernatant. Dendritic cells (1×10⁶/ml) were stimulated with medium only, LPS (1 μg/ml) alone or with FHA (10-5000 ng/ml). Supernatants were removed after 24 hours and IL-10 concentrations determined by two-site ELISA;

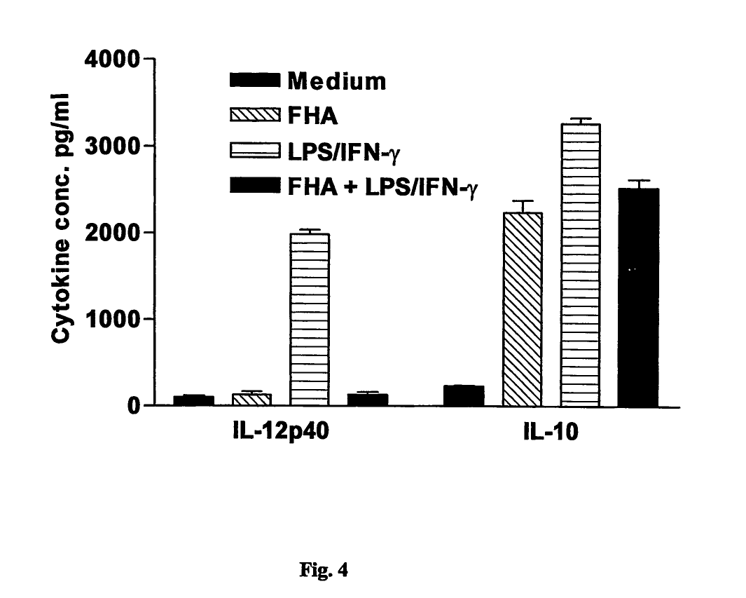

FIG. 4 is a graph showing that immunomodulatory activity of a freeze-dried preparation following long term storage. Macrophages were recovered from the peritoneal cavity of normal BALB/c mice. A preparation of FHA that had been freeze dried 5 years earlier and reconstituted in PBS prior to addition to macrophages (1×10⁶). Cells were stimulated with FHA (5 μg/ml), LPS (1 μg/ml) and IFN-γ (20 ng/ml) or LPS (1 μg/ml) and IFN-γ (20 ng/ml) in the presence of FHA (5 μg/ml). Supernatants were removed after 24 hours and IL-10 and IL-12p40 concentrations determined by two-site ELISA;

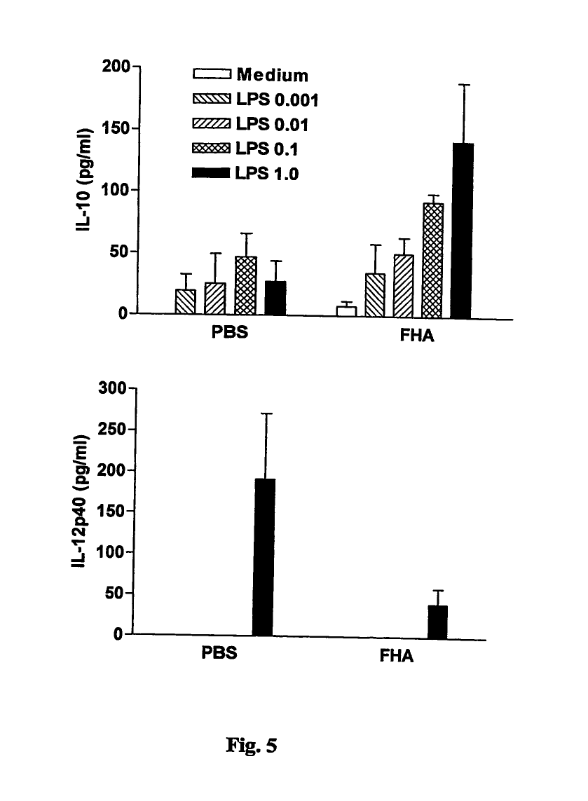

FIG. 5 is a graph showing enhanced IL-10 and suppressed IL-12 production by spleen cells from mice injected with FHA. Normal BALB/c mice were injected s.c. with FHA (10 μg/mouse) or PBS, 24 hours later spleen were removed and spleen cells stimulate with LPS (0.001-1.0 μg/ml or with medium only. Supernatants were removed after 24 hours and IL-10 and IL-12p40 concentrations determined by two-site ELISA.

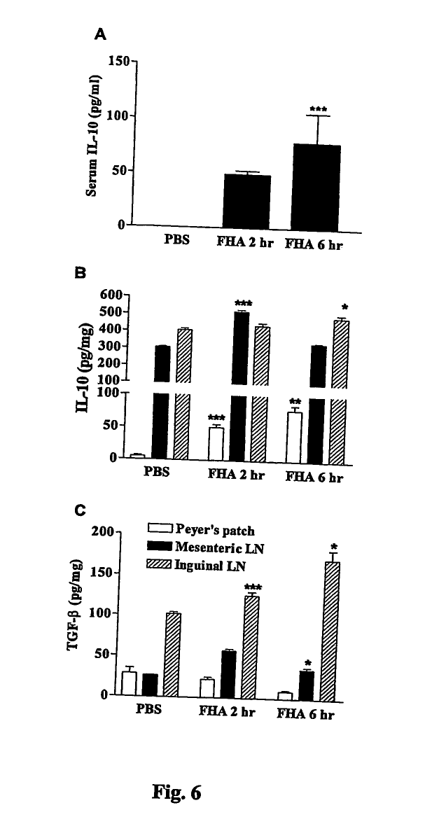

FIG. 6 is a graph showing that FHA induces production of the anti-inflammatory cytokines, IL-10 and TGF-β, in vivo. Normal BALB/c mice were injected s.c. with FHA (10 μg/mouse) or PBS, 2 or 6 hours later inguinal lymph nodes, mesenteric lymph nodes, peyer's patches and serum was recovered. IL-10 protein concentrations were determined in serum (A). IL-10 (B) and TGF-α (C) protein determined in homogenised lymphoid tissue by two-site ELISA. Statistically significant differences compared with PBS-treated mice: * P<0.05, *** P<0.01 and *** P<0.001.

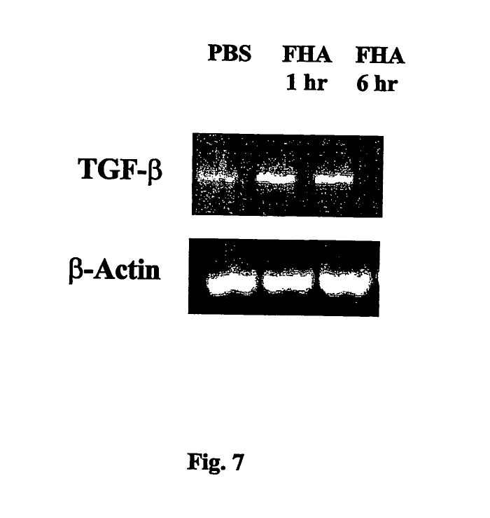

FIG. 7 are PCR results showing that FHA induces expression of TGF-β mRNA in vivo. BALB/c mice were injected s.c. with FHA (10 μg/mouse) or PBS and the inguinal lymph nodes removed 1 or 6 hours later. Lymph nodes were homogenized, total RNA extracted and TGF-β mRNA expression determined by RT-PCR;

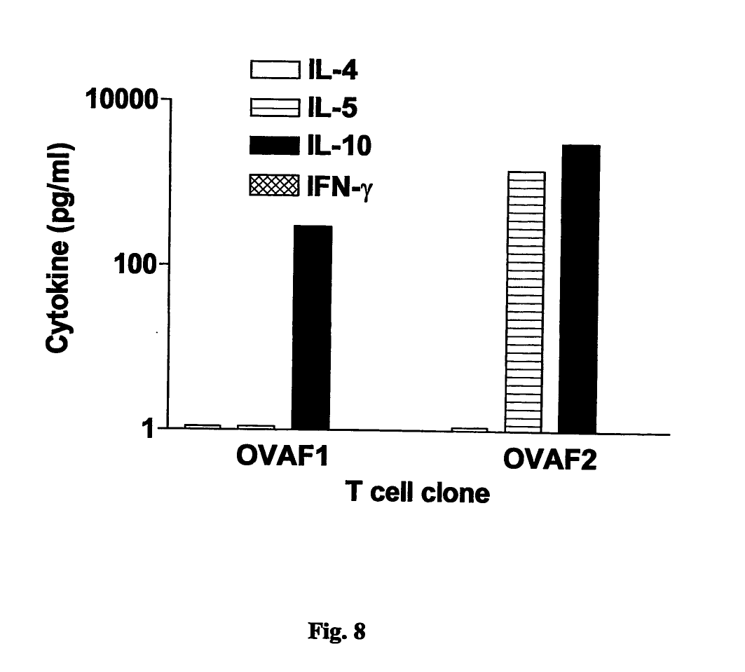

FIG. 8 is a graph showing that co-administration of FHA with a foreign antigen stimulates regulatory T cells specific for the co-administered antigen. BALB/c mice were immunized intranasally (day 0 and day 20) with an ovalbumin (OVA) peptide (323-339) (50 μg/mouse) and FHA (5 μg/mouse). The spleens were removed 7 days after the last immunization and re-cultured in vitro with OVA peptide. OVA-specific T cell lines were established from these cultures and then cloned by limiting dilution. T cell lines/clones were stimulated with OVA peptide and antigen presenting cells (irradiated syngenic spleen cells). Supernatants were removed after 2 days and IL-4, IL-5, IL-10 and TNF-α concentrations determined by 2-site ELISA;

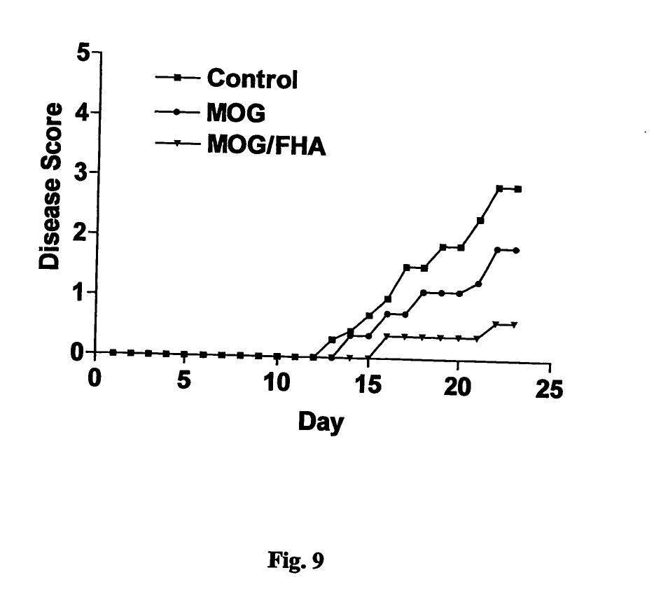

FIG. 9 is a graph showing the effect of immunization with myelin oligodendrocyte (MOG) peptides with FHA on the disease progression (average disease index) in experimental autoimmune encephalomyelitis (EAE), a murine model for multiple sclerosis. Mice were immunized subcutaneously (s.c.) with 50 μg MOG peptide (residues 35-55) and 5.0 μg FHA in phosphate buffered saline. This was repeated 21 days later. Control mice received MOG peptide or saline only. 7 days after the second immunization, EAE was induced by s.c. administration of 150 μg MOG peptide emulsified in complete Freund's adjuvant, supplemented with 5 mg/ml Mycobacteria tuberculosis intraperitoneal (i.p.) injection of 500 ng pertussis toxin, followed 2 days later by a second i.p. injection with 500 ng pertussis toxin. Mice were assessed daily for clinical signs of EAE, and scored as follows: 1=tail paralysis, 2=wobbly gait, 3=hind limb weakness, 4=hind limb paralysis, 5=complete paralysis of hind and fore limbs, 6=death. The disease index was calculated by adding all daily average disease scores, dividing the average day of onset, and multiplying by 100;

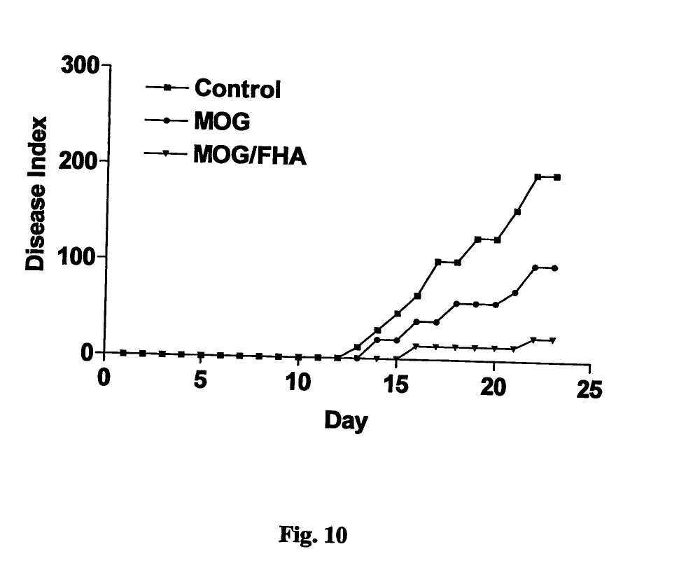

FIG. 10 is a graph showing the effect of immunization with FHA and MOG peptide on average disease score over time in experimental autoimmune encephalomyelitis (EAE);

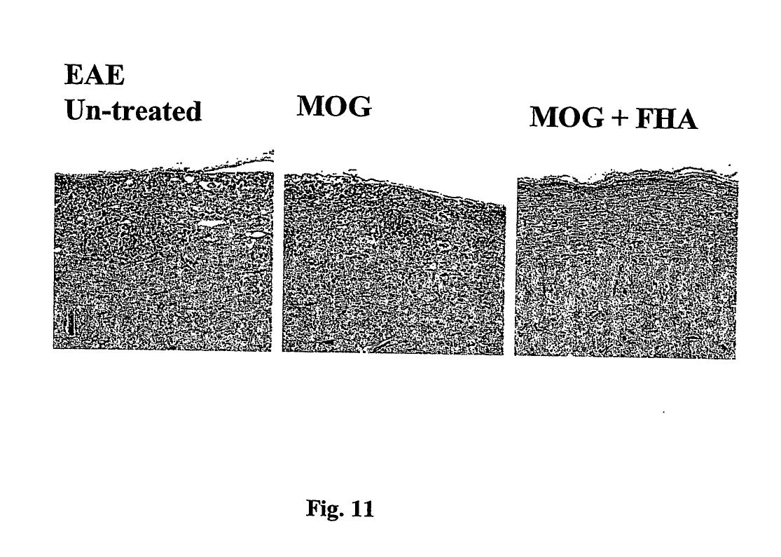

FIG. 11 is a micrograph showing histopathology section of spinal cords of mice after induction of EAE (untreated) or after immunization with myelin oligodendrocyte peptide (MOG) or MOG peptide+FHA (MOG +FHA). EAE was induced and mice immunized as described in FIG. 8, sections of spinal cord were removed from mice 23 days after induction of EAE and stained with haematoylin and eosin. The EAE induced in un-treated and MOG-immunized mice is severe with a pronounced mononuclear cell infiltrate; immunization with MOG and FHA prevents mononuclear cell infiltrate, encephalitis, pervascular cuffing and demyelination. In the EAE untreated, superficial white matter tracts and leptomeninges show myelin vacuolation, axonal degeneration and infiltration with lymphocytes and macrophages. In the MOG-immunized mouse, lesions are similar to those in the untreated EAE. In the FHA+MOG-immunized mouse, white matter tracts appear normal and small numbers of leucocytes are confined to the leptomeninges;

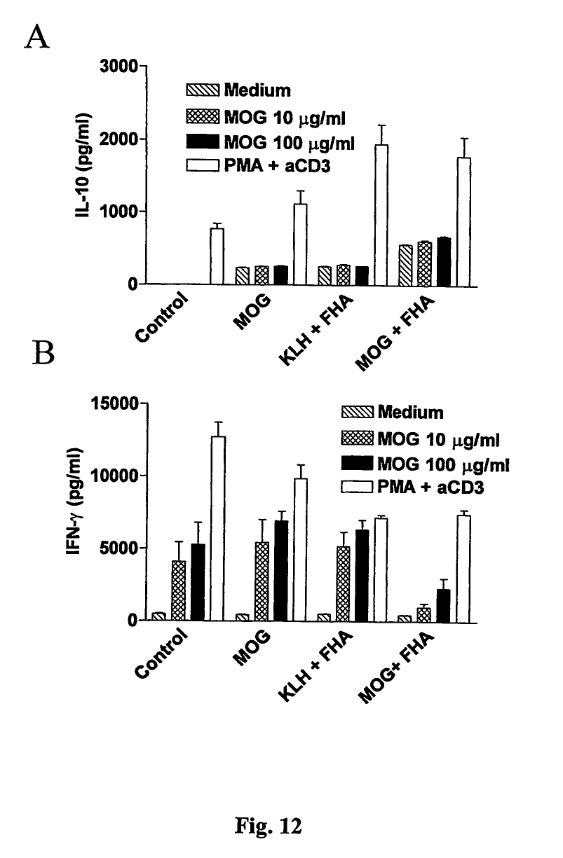

FIG. 12 are graphs showing that prophylactic treatment with MOG and FHA prior to induction of EAE suppress MOG-specific IFN-γ production. C57BL/6 mice were immunized subcutaneously with PBS only or MOG (50 μg) and FHA (5 μg) at days 0 and 21. EAE was induced 7 days later by subcutaneous administration of 150 μg MOG 35-55 peptide in Complete Freund's adjuvant, supplemented with 1 mg Mycobacteria tuberculosis and intraperitoneal (i.p.) administration of 500 ng pertussis toxin, followed 2 days later by a second i.p. injection of 500 ng pertussis toxin. Spleen cells were recovered 23 days after induction of EAE and re-stimulated in vitro with MOG peptide 35-55 (10 or 25 μg/ml), anti-CD3 and PMA or medium only. After 3 days of culture supernatants were removed and IFN-γ (FIG. 12B) and IL-10 (FIG. 12A) concentrations measured by two-site ELISA;

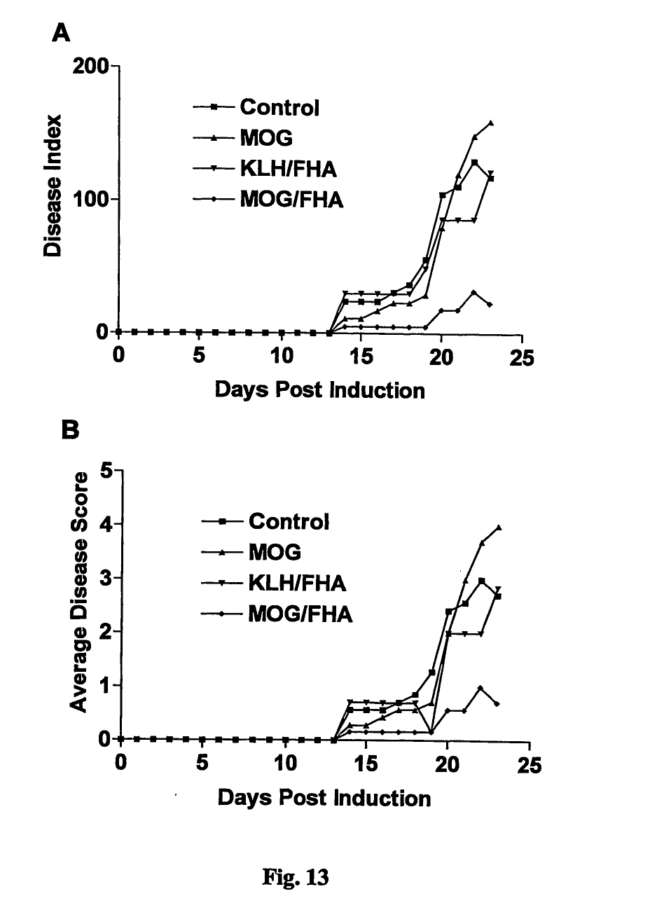

FIG. 13 are graphs showing that preventative immunotherapy with FHA as adjuvant is dependent on co-immunization with the self-antigen. (A) shows the disease index and (B) shows the average disease score. C57BL/6 mice were immunised subcutaneously (s.c.) with PBS, MOG (50 μg), keyhole limpet haemocyanin (KLH) (a model foreign antigen), (50 μg) and FHA (5 μg) or MOG (50 μg) and FHA (5 μg) at days 0 and 21. EAE was induced 7 days later with 150 μg of MOG 35-55 in complete Freund's adjuvant (CFA) s.c. and 500 ng pertussis toxin (PT) intraperitoneally (i.p.). PT (500 ng) was also administered i.p. 2 days later. Disease index and average disease score calculated as indicated in FIG. 9 (n=6 per group);

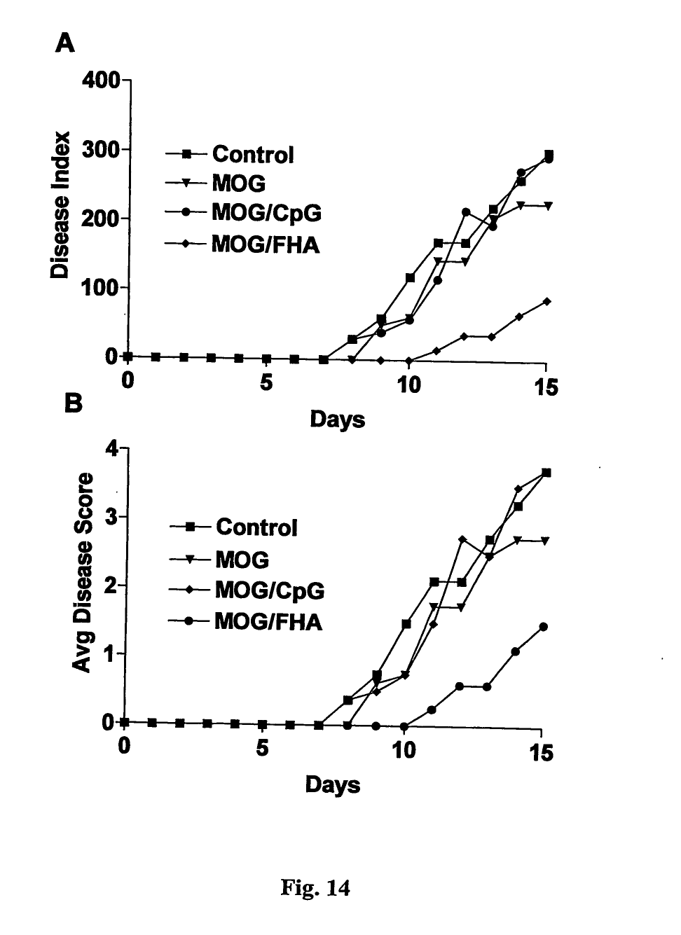

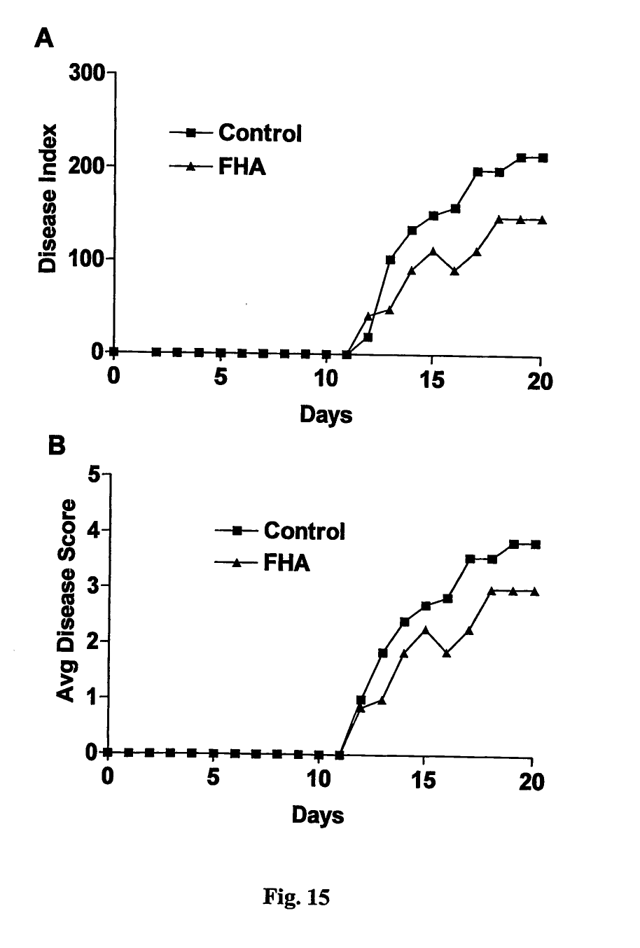

FIG. 14 are graphs showing that CD4⁺ T cells from mice immunised with MOG and FHA confer protection against EAE following transfer into recipient mice. Donor mice received two subcutaneous (s.c.) injections of either MOG (50 μg) or MOG (50 μg) and FHA (5 μg) or MOG (50 μg) and CpG (25 μg) at days 0 and 21. Seven days after the second immunization, splenic cells were harvested and purified CD4⁺ T cell purified using a CD4⁺ T cell purification column from R&D Systems. Recipient mice received 0.9×10⁶ CD4⁺ T cells intravenously from mice immunized with MOG, MOG and FHA or MOG and CpG 7 days after induction of EAE. Control mice received no cells. EAE was induced and day of onset, disease score (FIG. 15B) and disease index (FIG. 14A) were calculated as described in FIG. 9. (n=8 per group);

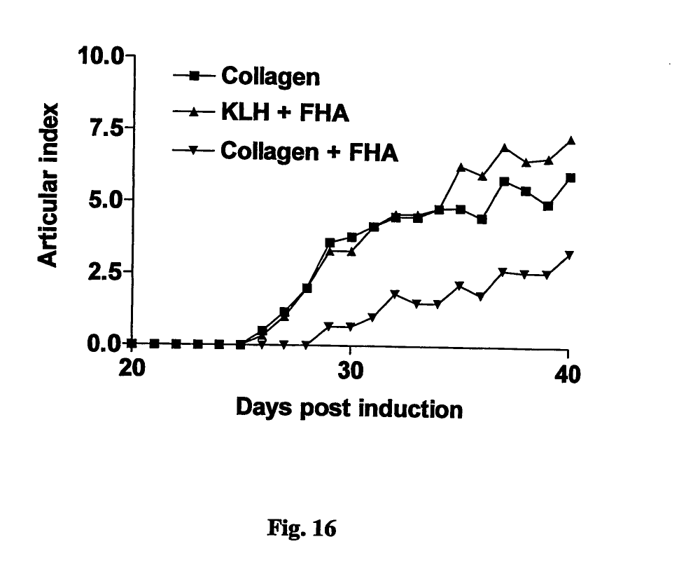

FIG. 15 are graphs showing that therapy with a single dose of FHA reduced the clinical signs EAE. Disease index from C57BL/6 mice immunised with 150 μg MOG35-55 in complete Freund's adjuvant (CFA) subcutaneously with 500 ng pertussis toxin (PT) administered intraperitoneally (i.p.). pertussis toxin (PT) was administered i.p. again on day 2. Mice were given one s.c. injection of 5 μg FHA on day 9. Disease index (FIG. 16A) and disease scores (FIG. 16B) were calculated as described in FIGS. 9;

FIG. 16 is a graph showing that immunization with FHA and type II collagen inhibits the development of collagen-induced arthritis in mice. Male DBA/1 mice were immunised with either type II collagen, keyhole limpet haeomocyanin (KLH) and FHA or collagen and FHA at days 0 and 21. Seven days later, arthritis was induced by intradermal injection of type II collagen in complete Freund's adjuvant (CFA). Mice were boosted with an intraperitoneal injection of collagen in PBS 21 days later. The disease course was monitored for 40 days. Mice were scored daily according to the following score. 0-normal; 1-erythema; 2-erythema and swelling; 3-loss of function. The articular index was calculated by adding the score for all four paws of each mouse and calculating the group average (n=6 per group);

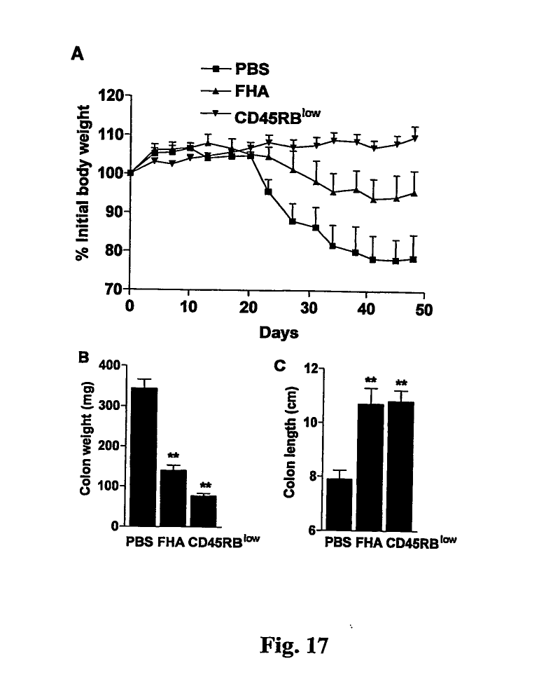

FIG. 17 are graphs showing the effect of s.c. administration of FHA on the development of intestinal inflammation in a murine colitis model. Three groups of SCID mice were injected i.p. with 1×10⁵ CD4⁺CD45RB^{high} T-cells. One group of mice were injected s.c. with PBS every two weeks, one group of mice was treated with 10 μg FHA s.c. every two weeks and the third group of mice were injected i.p. with 4×10⁵ CD4⁺CD45RB^{low} T-cells on day 0. (FIG. 17A) Body weight was recorded twice weekly. Treatment with FHA or injection of CD4⁺CD45RB^{low} T-cells significantly decreased wasting disease induced by the injection of CD4⁺CD45RB^{high} T-cells (P<0.01 and P<0.0001 respectively using a single factor ANOVA, CI 95%). (FIG. 17B). At the end of the experiment (day 56) the colon weights and colon lengths were measured. ** Indicates statistically significant differences compared with PBS-treated mice (P<0.01). Administration of CD45RB^{hi} cells was associated with the development of severe intestinal inflammation in SCID mice, which was accompanied by severe weight loss. Transfer of CD45RB^{low} cells prevented inflammation and weight loss. Sub-cutaneous therapy with FHA prevented colon inflammation and weight loss. FHA treated mice had a marked reduction of intestinal inflammation, significantly (P<0.01) reduced colon weights and less colon shrinkage than control CD45RB^{hi} transferred mice mice given no treatment;

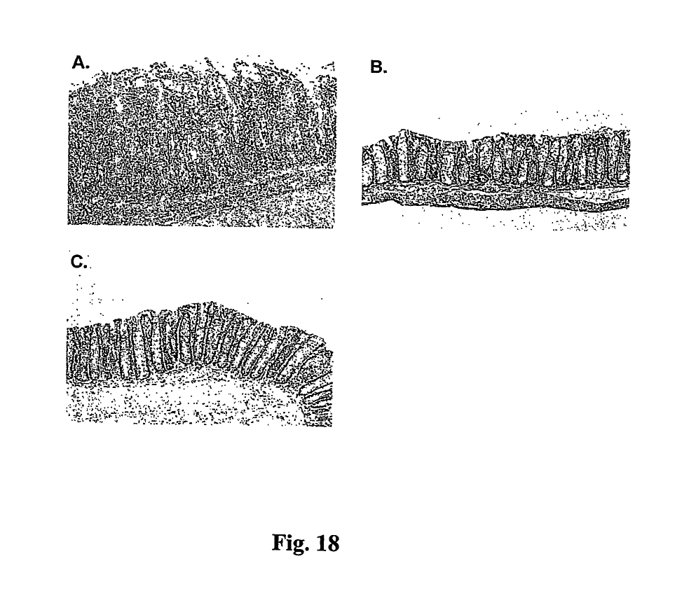

FIG. 18 are micrographs showings histological evidence that FHA can prevent colon inflammation. Colitis was induced and mice treated as described FIG. 17. Colons were removed on day 56, sections cut, mounted and stained with H&E. A) SCID mice injected i.p. with 1×10⁵ CD45RB^{high} T cells and injected with PBS every 2 weeks from day 0. B) SCID mice injected i.p. with 1×10⁵ CD45RB^{high} and 4×10⁵ CD45RB^{low} T cells on day 0. C) SCID mice injected i.p. with 1×10⁵ CD45RB^{high} naïve T cells and injected with FHA s.c. every two weeks;

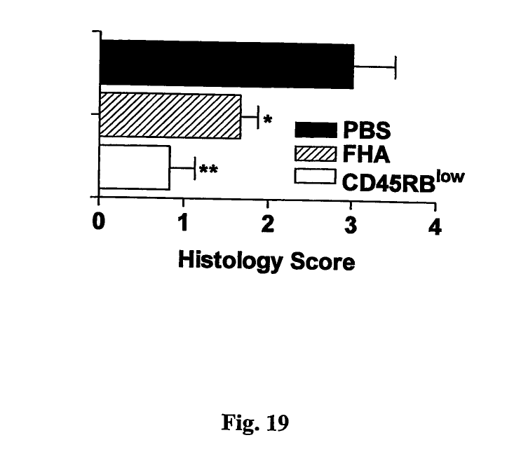

FIG. 19 is a graph showing that FHA significantly reduced colon inflammation in an experimental colitis model. Colitis was induced and mice treated as described in FIG. 18. Colons were scored for different inflammatory characteristics on a scale from 0 to 4. Statistically significant differences compared with PBS-treated mice: * P<0.05 and ** P<0.01;

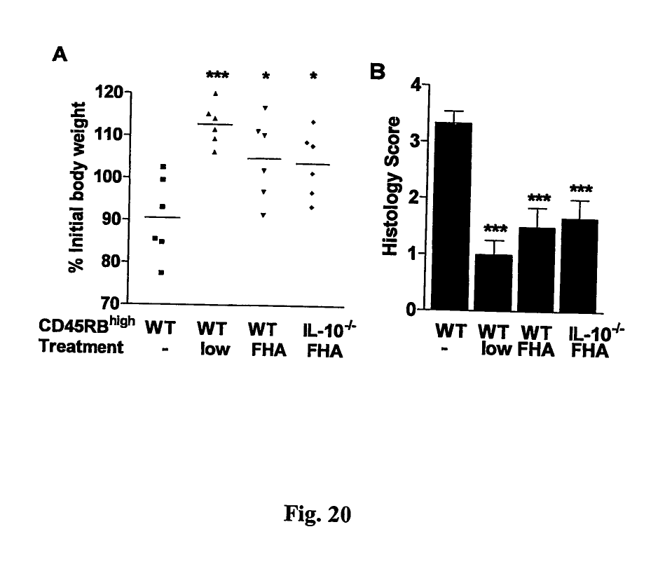

FIG. 20 are graphs showing the role of T cell-derived IL-10 in the protective effect of FHA on induction of T-cell mediated colitis in mice. Four experimental group were employed as follows:
Group 1: SCID mice injected with 1×10⁵ CD45RB^{high} T-cells, no treatment
Group 2: SCID mice injected with 1×10⁵ CD45RB^{high} and 4×10⁵ CD45RB^{low} T-cells
Group 3: SCID mice injected with 1×10⁵ CD45RB^{high} T-cells and treated with 10 μg FHA s.c. every 2 weeks.
Group 4: SCID mice injected with 1×10⁵ IL-10-defective CD45RB^{high} T-cells and treated with 10 μg FHA s.c. every 2 weeks.

(A) Body weights was recorded twice weekly. (B) At the end of the experiment (day 56) the colon were removed prepared for histology and scored for different inflammatory characteristics on a scale from 0 to 4. Statistically significant differences compared with PBS-treated mice: * P<0.05 and *** P<0.001;

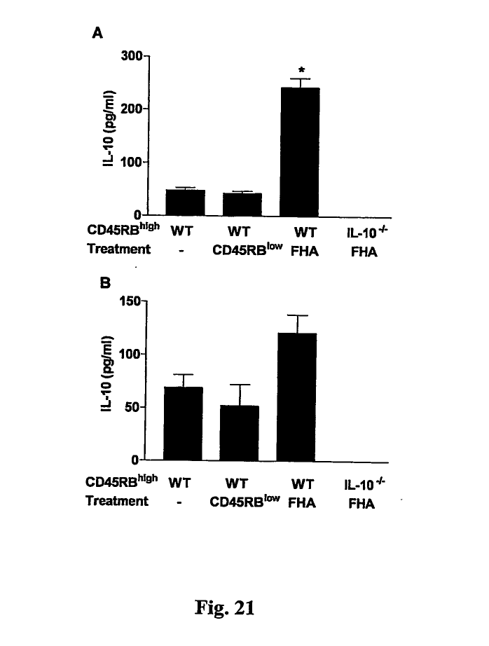

FIG. 21 is a graph showing enhanced IL-10 production by spleen cells from SCID mice injected with CD4⁺CD45RB^{high} cells and treated with FHA. Four experimental groups of mice were employed as described in FIG. 20. Spleen cells were recovered from the 4 groups of mice 8 weeks after treatment. CD4⁺ T cell were purified from the bulk spleen cells using a FACS. Unseparated spleen cells or purified CD4⁺ T cells were stimulated with anti-CD3 and anti-CD28. IL-10 concentrations were tested in supernatants, removed 2 days after the second stimulation. Results are mean ±SD for triplicate cultures. The results show significant enhancement of IL-10 production by splenic T cells (p<0.05) and purified CD4⁺ T cells (not statistically significant) from FHA-treated mice; and

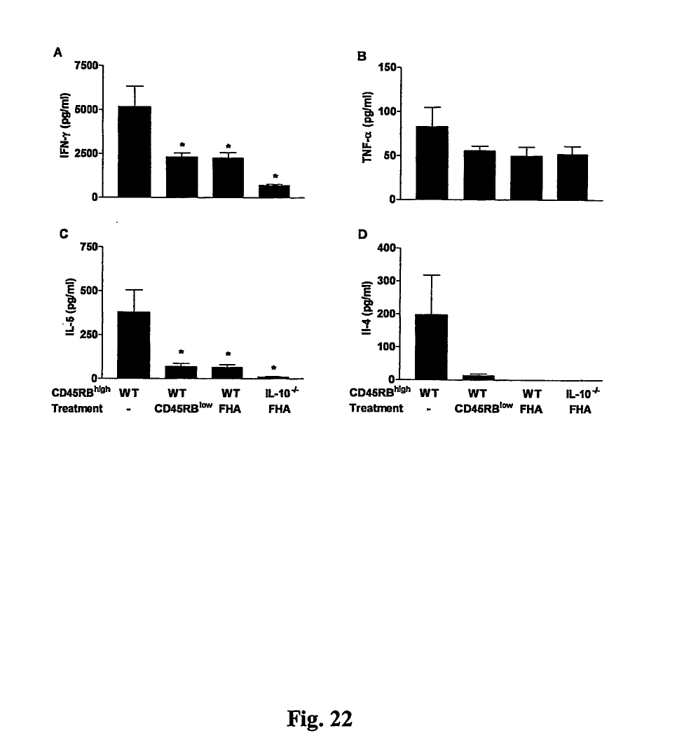

FIG. 22 are graphs showing reduced pro-inflammatory cytokine production by spleen cells from FHA-treated mice injected with CD4⁺CD45RB^{high} cells. Spleen cells were recovered from SCID mice 8 weeks after injection with CD4⁺CD45RB^{high} cells and treatment as described in FIG. 20. Spleen cells were stimulated with anti-CD3 and anti-CD28. IFN-γ (A), TNF-α (B) IL-4 (C) and IL-5 (D) concentrations were tested in supernatants, removed 2 days after the second stimulation. The results show significant suppression of IFN-γ and IL-5 production by splenic T cells (p<0.05). Results are mean ±SD for triplicate cultures.

### DETAILED DESCRIPTION

We have found that filamentous haemagglutinin (FHA) from Bordetella pertussis may be used as a therapy or as an immunomodulator in a vaccine against immune-mediated diseases, including in a vaccine against autoimmune disease. Parenteral immunisation of mice with the myelin oligodendrocyte glycoprotein (MOG) synthetic peptide in the presence of FHA was found to prevent the development of disease symptoms and pathology in experimental allergic encephalomyelitis (EAE), a murine model for multiple sclerosis. Immunisation with self or foreign antigens in the presence of FHA promotes the induction of regulatory T cells specific for the bystander antigen and these T cells appear to be capable of preventing self-reactive immune responses leading to autoimmune conditions.

Current approaches for the treatment of multiple sclerosis have focused on therapeutic strategies aimed at reducing inflammation in the brain of individuals who have already started to develop disease symptoms.

We have found that FHA may be used to prevent the onset of clinical signs of EAE by inducing memory T cells with suppressor activity and are specific for myelin proteins.

We also found that sub-cutaneous administration of FHA reduced the intestinal inflammation, reduced colon weight gain and shrinkage and prevented weight loss induced in SCID mice by transfer of naïve CD45RB^{hi} cells. This indicates that FHA can prevent the development of autoimmune diseases, possibly by the induction of regulatory T cells or by the production of innate IL-10 and TGF-β, which promotes the induction of regulatory T cells or has a direct suppressive effects on the immune responses that mediate autoimmune diseases.

Co-administration of FHA with a foreign antigen stimulates regulatory T cells specific for the co-administered antigen. FHA was found to promote the induction of T cells that secrete IL-5 and IL-10, but not IL-4 or TNF-α indicating that it directs the induction of type 1 regulatory T (TR1) cells in vivo. (FIG. 8)

We found that mice immunised with self-antigen and FHA were conferred with protection against EAE following transfer into recipient mice. Therapy with a single dose of FHA reduced clinical signs of EAE.

One pharmaceutical product that may be envisaged comprises FHA in combination with a pharmaceutically acceptable antigen, which may be for example a self-antigen or a foreign antigen, or in combination with a peptide thereof, of for example a self-antigen or a foreign antigen. Such combination products would have a beneficial effect in that the FHA works in synergy with the component with which it is combined. Such products may be prepared using processes commonly known to the person skilled in the art.

We have found that FHA production of IL-10 and suppression of IL-12 and IFN-γ is enhanced by the presence of a toll-like receptor ligand (TLR ligand), such as LPS Other TLR ligands may be selected from any one or more of Pam3Cys, CpG motifs, dsRNA or Poly (I:C). FHA in combination with a pharmaceutically acceptable TLR ligand would appear to have an enhanced immunomodulatory effect. It is envisaged that FHA in combination with a pharmaceutically acceptable TLR ligand and a self-antigen would also provide an enhanced immunomodulatory and/or anti-inflammatory effect.

FHA has already been approved for use in humans and is currently a component of several acellular pertussis vaccines, where it is absorbed to aluminium hydroxide.

FHA or derivatives thereof may be used in the treatment of, or as a therapy or as a component of a vaccine in the prevention of immune mediated diseases, including but not limited to multiple sclerosis, Crohn's disease, inflammatory bowel disease, type 1 diabetes, rheumatoid arthritis or psoriasis.

FHA or derivatives thereof may also be used in the treatment of, or as a therapy or as a component of a vaccine in the prevention of an immune-mediated disorder selected from any one or more of diabetes mellitus, arthritis (including rheumatoid arthritis, juvenile rheumatoid arthritis, osteoarthritis, psoriatic arthritis), myasthenia gravis, systemic lupus erythematosis, autoimmune thyroiditis, dermatitis (including atopic dermatitis and eczematous dermatitis), Sjogren's Syndrome, including keratoconjunctivitis sicca secondary to Sjogren's Syndrome, alopecia areata, allergic responses due to arthropod bite reactions, aphthous ulcer, iritis, conjunctivitis, keratoconjunctivitis, ulcerative colitis, asthma, allergic asthma, cutaneous lupus erythematosus, scieroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, interstitial lung fibrosis, Alzheimer's disease or coeliac disease.

FHA or derivatives thereof may also be used in treatment of, or as a component of a vaccine in the prevention of asthma or atopic diseases.

Many of the diseases detailed above have no satisfactory treatment and in most cases steroids and non-steroidal anti-inflammatory drugs are employed. However, these drugs are non-specific drugs and have side effects. More recently drugs that inhibit key inflammatory cytokines, in particular tumour necrosis factor (TNF)-α, have been developed. These include antibodies or soluble TNF receptors that are effective against certain autoimmune diseases, but are associated with side effects (including recurrent tuberculosis) and are limited to diseases where TNF-α is the key mediator of pathology. Another therapeutic approach is the direct administration of anti-inflammatory cytokines (e.g. IL-10), but this is compromised by the short half-life of the cytokines in vivo. Alternative strategies could employ agents that induce anti-inflammatory cytokines, such as IL-10, which will have a direct immunosuppressive effect in vivo.

Molecules that stimulate anti-inflammatory cytokines and inhibit pro-inflammatory cytokine production from cells of the innate immune system and promote the induction of suppressor or regulatory T cells, have the potential to limit inflammatory and Th1-mediated immune responses. FHA has the potential to drive innate and adaptive IL-10 and thereby act as an immunotherapeutic drug or as an immunomodulator or adjuvant for vaccines to prevent immune mediated disease.

Chronic intestinal inflammation in Crohn's disease is associated with excessive production of the T helper 1 (Th1) and pro-inflammatory cytokines, IFN-γ, TNF-α and IL-12 (9) There is evidence these harmful inflammatory and tissue destructive immune responses may result from a defect in anti-inflammatory responses or loss of tolerance to commensal bacterial antigens. The production of anti-inflammatory cytokines, IL-10 and TGF-β in response to TLR-2 ligands and enteric bacteria is significantly reduced and Th1 responses enhanced in Crohn's disease patients with a mutation in NOD-2, an intracellular pathogen recognition receptor (PRR) for muramyl dipeptide from bacterial peptidoglycan (10, 11). Alternatively, immune-mediated diseases can arise through defective counter-regulation of inflammatory responses as a result of a failure to generate Tr cells. (8). Transfer of Tr cells has been shown to confer protection against intestinal inflammation in animal model of colitis. (12). Therefore strategies that target the induction of anti-inflammatory cytokines and Tr cells in vivo have considerable promise in the development of new therapies against Crohn's disease.

Immuno-suppressive therapies are used for the treatment Crohn's disease, including TNF antibodies or TNF antagonists and azathioprine. To our current knowledge these therapies mediate their effects through direct inhibition of a key pro-inflammatory mediator or the induction of apoptosis of activated T-cells and not through modulation of DC function. (13, 14) Direct administration of the anti-inflammatory cytokine, IL-10 has also been explored, but IL-10 has a short half life in vivo and clinical trails showed only modest therapeutic benefit (15). Bacteria genetically engineered to express IL-10 have also been shown to reduce colitis in animal models (16). An alternative approach adopted in this invention was to stimulate production anti-inflammatory cytokines in vivo, by targeting appropriate host cells with pathogen-derived immunomodulatory molecules.

FHA has the capacity to modulate innate immune cell function to produce anti-inflammatory cytokines, which in turn may be amplified through the secondary induction of Tr-cells. In the present invention, injection of FHA led to an immediate (2-6 hrs) induction of IL-10 and TGF-β in regional and distant lymph nodes and Peyer's patches, suggesting that FHA modulates tissue DCs or macrophages to migrate to lymph nodes and secondary lymphoid tissues and to initiate anti-inflammatory responses. Both IL-10 and TGF-β are crucial in the prevention of T-cell mediated colitis (12, 17) and are likely to be responsible for the presence of IL-10 producing T cells detected in the spleen of FHA-treated severe combined immunodeficient (SCID) mice. FHA-treated mice displayed decreased colitis, enhanced spleen cell IL-10 and concomitant reduction in pro-inflammatory Th1-type cytokines (IFN-γ and TNF-α) compared to PBS-treated mice. Interestingly, FHA induced the same levels of protection against colitis in SCID mice transferred with IL-10^{-/-} and wildtype CD4⁺CD45RB^{high} T cells, suggesting that protection was not mediated by T-cell derived IL-10. This does not rule out a role for Tr-cells in protection, as Tr-cells can also suppress immune responses through TGF-β production and through cell-to-cell contact.

Increased IL-10 production by splenic T cells indicates that FHA-stimulated immunomodulatory responses are not limited to local tissues but are also manifested at distant sites, including the intestine.

FHA binds to CD11b/CD18 on DCs and modulates the function of these cells to generate IL-10 producing Tr-cells that in turn are not dependent on their IL-10 producing capacity to suppress the development of colitis. Immunomodulation of DCs and as a consequence the activation of Tr-cells alone or collectively may provide useful strategies for the prevention of colitis. FHA has considerable potential as a therapy for Crohn's disease.

The invention will be more clearly understood by the following examples.

### EXAMPLES

FHA Purification

Bordetella pertussis was grown for 3 days on Bordet-Gengou agar plates. The colonies, which were hemolytic, were used to start a liquid preculture (30 ml) in Stainer-Scholte (SS) medium, supplemented with dimethyl-beta cyclodextrin (CDX; purchased from Sigma) at a final concentration of 0.5 g per liter (CDX induces the release of FHA from the bacterial surface). This pre-culture was grown overnight at 37° C. under agitation and used to inoculate a large cultures (250 ml of SS medium in 1-L flasks). This culture was grown at 37° C. under agitation for 36-48 hours. Once the plateau phase was reached (determined by measuring optical density of the culture), the cells in culture medium was centrifuged at 7000 rpm for 20 min at 4° C. and the supernatant collect.

The FHA was purified from the supernatant using FPLC with a matrix of heparin-sepharose column (Amersham) equilibrated with PBS pH 7.4. After loading the sample, the column was washed with PBS and eluted with PBS supplemented with 0.5 M NaCl at room temperature using a flow rate of 2 ml/minute. The fractions with the peak elution contained the FHA. Contaminating LPS and was removed on endotoxin-removal columns (Detoxi-Gel™ endotoxin removing gel; Pierce, Rockford, Ill., USA). Following this step, endotoxin was undetectable in the preparation using the chromogenic limulus amebocyte lysate (LAL) assay (Bio Whittaker, Walkersville, Md., USA).

In preparations of FHA there are residual amounts of endotoxin or lipopolysaccharide (LPS). This residual amount of endotoxin is however very low. Typically the concentration of contaminating endotoxin is less than 300 pg/μg of protein.

To date commercially available preparations of FHA and FHA preparations discussed in the art have a considerably higher concentration of contaminating endotoxin. We have added additional steps in the purification of FHA to remove contaminating LPS as follows:

The following dialysis steps are performed on the protein preparation: 1) The FHA protein preparation is dialysed for 1 hr and then overnight against a fresh preparation of 8M Urea, 2) The FHA protein preparation is dialysed for 1 hr and then overnight against a fresh preparation of 4M Urea and 3) The FHA Protein is dialysed for 1 hr and then overnight against a fresh preparation of 1M Urea.

The purpose of these initial steps is to ensure complete denaturation of the protein. This exposes LPS bound to the inner hydrophobic regions of FHA. The concentration of Urea must be gradually reduced, otherwise irreparable damage is done to the protein and it will not refold correctly. The residual LPS is then removed as follows:
i) Wash an Etoxate column (Pierce) with 5 ml of endotoxin free water containing 1% sodium deoxycholate
ii) Wash column with 5 ml endotoxin free water.
iii) Add the protein solution
iv) Wash the column again with 5 ml 1% sodium deoxycholate and collect 1-2 ml fractions
v) Measure protein concentrations of fractions by testing optical density at 600 nm to determine the fraction with highest protein concentration

Sodium deoxycholate removes all LPS bound to the etoxate column. However, the majority of non-protein bound LPS will be eluted off in a separate fraction to protein bound LPS due to differences in density.

We have shown that the stimulation of IL-10 production by FHA appears to be augmented by LPS signalling through Toll-like receptor-4 (TLR-4). The amount of LPS required to augment the effect of FHA is considerably higher than the residual amount of LPS that may be present in a purified preparation of FHA. Treatment of DC with LPS alone at a concentration present in the FHA preparation (0.2 ng/ml) failed to activate DC in vitro. FIG. 2 shows that FHA alone stimulates IL-10 production by peritoneal macrophages from C3H/HeN and TLR-4-defective C3H/HeJ mice, indicating that IL-10 production is not dependant on LPS but is enhanced by LPS or on signalling though TLR-4. Addition of LPS enhanced FHA-induced IL-10 production by peritoneal macrophages from C3H/HeN mice, but has little affect on FHA-induced IL-10 from C3H/HeJ mice. FHA also stimulates low levels of IL-10 production by dendritic cells from C3H/HeN mice, but not from TLR-4-defective C3H/HeJ mice. FHA-induced IL-10 production from dendritic cells from C3H/HeN mice is enhanced by addition of LPS.

FIG. 3 shows that FHA at a concentration of 5 μg/ml enhances LPS-induced IL-10 production from dendritic cells. In the absence of TLR-4 signalling in C3H/HeJ mice, FHA does not stimulate IL-10 production from DC, but at concentration of 1-5 μg/ml does enhance LPS-induced IL-10 production. These results suggest that the efficacy of FHA as an immunomodulator may be enhanced by co-injection with a pharmaceutically acceptable TLR ligand.

FHA induces IL-10 production from human monocytes and inhibits LPS and IFN-γ induced IL-12 production (FIG. 1). Furthermore injection of FHA stimulates IL-10 and inhibits IL-12 production in vivo (FIG. 5).

Long term storage of FHA did not appear to affect the activity. FHA was found to stimulate IL-10 production and inhibit LPS and IFN-γ production by murine macrophages and this effect is maintained following long term storage of FHA as a freeze dried preparation (FIG. 4).

FIG. 6 shows that s.c injection of FHA in mice resulted in enhanced production of the anti-inflammatory cytokines, IL-10 and TGF-β, in lymph nodes within 1-2 hours of injection and that this effect persisted and is enhanced 6 hours after administration.

TGF-β was induced at the transcriptional level within 1 hour of s.c injection of FHA, an effect that is sustained for at least 6 hours (FIG. 7).

Murine Model for Multiple Sclerosis

Experimental autoimmune encephalomyelitis (EAE) is a murine model for multiple sclerosis. EAE is induced in C57BL/6 mice by s.c. administration of 150 μg MOG peptide emulsified in complete Freund's adjuvant, supplemented with 1 mg Mycobacteria tuberculosis intraperitoneal (i.p.) injection of 500 ng pertussis toxin, followed 2 days later by a second i.p. injection with 500 ng pertussis toxin. Mice develop symptoms of paralysis. In experiments to assess the effects of FHA as a adjuvant for a vaccine against autoimmune disease, mice were immunized subcutaneously (s.c.) with 50 μg MOG peptide (residues 35-55) and 5.0 μg FHA in phosphate buffered saline. This was repeated 21 days later. Control mice received MOG peptide or saline only. 7 days after the second immunization. Mice were assessed daily for clinical signs of EAE, and scored as follows: 1=tail paralysis, 2=wobbly gait, 3=hind limb weakness, 4=hind limb paralysis, 5=complete paralysis of hind and fore limbs, 6=death.

Table 1 shows the disease score and disease index results. The results indicate that the administration of FHA as an adjuvant significantly inhibits disease progression.

TABLE 1

| Immunization | | Day of | Mean Max | Disease Index |
|---|---|---|---|---|
| Group | Incidence | onset | Clinical Score | at day 23 |
| Control | 10/11 | 16.4 | 2.9 | 195 |
| MOG | 7/8 | 15 | 1.875 | 100 |
| MOG + FHA | 5/8 | 20.5 | 0.625 | 5 |

Incidence is the number of mice out of the number tested that develop any clinical symptoms of EAE. The disease index was calculated by adding all daily average disease scores, dividing the average day of onset, and multiplying by 100.

The effect of immunization with myelin oligodendrocyte (MOG) peptides with FHA on the disease progression (average disease index) in experimental autoimmune encephalomyelitis (EAE) is shown in FIG. 9. FIG. 10 shows the average disease score over time in an EAE model. Histology results clearly show the effect of immunisation with MOG and FHA (FIG. 11).

Spontaneous MOG-specific and PMA and anti-CD3-induced IL-10 is enhanced in mice immunized with MOG+FHA. In contrast, MOG-specific IFN-γ production is significantly reduced in mice immunized with MOG+FHA. FIG. 12 shows that prophylactic treatment with MOG and FHA prior to induction of EAE suppress MOG-specific IFN-γ production.

Preventative immunotherapy with FHA as immunomodulator is dependent on co-immunization with the self-antigen. Immunization with FHA and MOG prevents the development of EAE, whereas immunization with a control antigen (KLH) and FHA does not prevent EAE. This indicates that preventative immunotherapy with FHA (i.e. administered prior to induction of EAE) is dependant on co-administration with a self antigen (in this example the MOG peptide) (FIG. 13).

We found that CD4⁺ T cells from mice immunised with MOG and FHA confer protection against EAE following transfer into recipient mice (FIG. 14). The transfer of CD4⁺ T cells from mice immunized with MOG and FHA 7 days after induction of EAE prevents the development of EAE. In contrast CD4⁺ T cells from mice immunized with MOG alone or with MOG and CpG were not protective. This indicates that immunization with MOG and FHA induce a population of MOG-specific Tr cells, which suppress immune responses that lead to the development of EAE.

We found that therapy with a single dose of FHA reduced the clinical signs EAE (FIG. 15). Disease index and disease scores were calculated as described in FIGS. 9 and 10. A single dose of FHA after onset of EAE lessened the severity of disease.

Immunization with FHA and type II collagen was found to inhibit the development of collagen-induced arthritis in mice (FIG. 16). The severity of disease in mice immunised with collagen and FHA was reduced in comparison to mice immunised with KLH and FHA.

Murine Model for Colitis in Humans

Sub-cutaneous administration of FHA on the development of intestinal inflammation in a murine colitis model is shown in FIG. 17. CD45RB^{hi} naive T cells were injected into severe combined immunodeficient (SCID) mice. This results in the development of chronic colonic inflammation 6-8 weeks after injection. Sub-cutaneous therapy with FHA prevented colon inflammation and weight loss (FIG. 17A). FHA treated mice had a marked reduction of intestinal inflammation, reduced colon weights and less colon shrinkage than control CD45RB^{hi} transferred mice given no treatment (FIG. 17B and C).

Histology was characterized by influx of mononuclear cells in all layers of the intestinal wall, hyperplasia and decreased differentiation of intestinal epithelial cells (FIGS. 18 & 19).

Groups of 6 SCID mice were injected intravenously with CD45RB^{hi} naïve T cells alone or with CD45RB^{low} T cells or were injected with CD45RB^{hi} naïve T cells with FHA administered s.c. (10 μg/mouse 2 weeks apart). Body weight was recorded and mice were sacrificed after 8-12 weeks. Colon weights were recorded and histology was performed on hematoxylin and eosin stained sections of the colons (FIGS. 20A &B). Treatment with FHA prevents weight loss and significantly reduces colonic inflammation in SCID mice that have received naïve T cells from normal wildtype and IL-10-defective mice. This indicates that the protective effect of FHA is independent of T cell-derived IL-10.

Spleen cells were removed 8 weeks after induction of colitis in SCID mice injected with wild type CD4⁺CD45RB^{high} T cells treated with PBS, FHA or CD4⁺CD45RB^{low} T cells or injected with CD4⁺CD45RB^{high} T cells from IL-10^{-/-} mice and treated with FHA. Unseparated spleen cells and CD4⁺ T cells sorted from spleen preparations were stimulated twice with anti-CD3 and anti-C28 and cytokine concentrations determined in the supernatants 2 days after the second stimulation (FIGS. 21 and 22). Bulk spleen cells from wild type CD4⁺CD45RB^{high} injected mice treated with FHA produced significantly more IL-10 than mice treated with PBS or co-injected with CD4⁺CD45RB^{low} T cells (244±30 pg/ml vs. 43±7 pg/ml and 48±10 pg/ml) (FIG. 21). Moreover, IL-10 production was significantly higher in bulk spleen cells compared to CD4⁺ T cells in FHA treated mice (244±30 pg/ml vs. 121±31 pg/ml.

As expected, no IL-10 was detected when bulk spleen cells and CD4⁺ T cells from SCID mice injected with IL-10^{-/-} CD45RB^{high} T cells were stimulated with anti-CD3 and anti-CD28 (detection limit 20 pg/ml).

Relatively high concentrations of the pro-inflammatory Th1-type cytokines, IFN-γ (FIG. 22A), and TNF-α (FIG. 22B) were detected in spleen cells recovered from PBS-treated mice, these mice were also suffering from severe colitis. In contrast, spleen cells from FHA-treated SCID mice injected with T-cells from wild type or IL-10^{-/-} mice produced significantly lower concentrations of the TH1 cytokines, TNF-α and IFN-γ and the Th2 cytokines IL-4 and IL-5 (FIG. 22C and D) compared with spleen cells from PBS-treated SCID mice. These findings demonstrate that experimental colitis is associated with enhanced production of Th1-type cytokines, which can be detected in the spleen and that treatment with FHA suppresses pro-inflammatory cytokines, while it enhances IL-10

Dosage, Mode of Administration and Pharmaceutical Formulations

The invention includes methods of modulating an immune response in a mammal to a selected antigen, the method comprises administering to a mammal a therapeutic amount of an agent comprising FHA or a derivative or mutant or fragment or variant or peptide thereof or products of cells activated by these materials or administering a therapeutic amount of an agent comprising FHA or derivative or mutant or fragment or variant or peptide thereof and an antigen or FHA and a pharmaceutically acceptable toll-like receptor (TLR) ligand.

The compositions for administration may be prepared as injectables, either as liquid solutions or suspensions; solid forms suitable for solution in or suspension in, liquid prior to infection can also be prepared. The preparation can also be emulsified, or the composition encapsulated in liposomes. The active immunogenic ingredients are often mixed with carriers which are pharmaceutically acceptable and compatible with the active ingredient. The term “pharmaceutically acceptable carrier” refers to a carrier that does not cause an allergic reaction or other untoward effect in subjects to whom it is administered. Suitable pharmaceutically acceptable carriers include, for example, one or more of water, saline, phosphate buffered saline, dextrose, glycerol, ethanol, or the like and combinations thereof. In addition, if desired, the immunomodulator/formulation can contain minor amounts of auxiliary substances such as wetting or emulsifying agents, pH buffering agents, and/or adjuvants which enhance the effectiveness of the formulation/immunomodulator.

Compositions of the invention may be administered parenterally, by injection, for example, either subcutaneously, epicutaneously or intramuscularly. Additional formulations which are suitable for other modes of administration include suppositories, and in some cases, oral formulations, nasal formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25-70%.

The compositions of the invention may be formulated into the immunomodulator compositions as neutral or salt forms. Pharmaceutically acceptable salts include the acid addition salts (formed with free amino groups of the peptide) and which are formed with inorganic acids such as, for example, hydrochloric or phosphoric acids, or with organic acids such as acetic, oxalic, tartaric, maleic, and the like. Salts formed with the free carboxyl groups can also be derived from inorganic bases such as, for example, sodium, potassium, ammonium, calcium, or ferric hydroides, and such organic bases as isopropylamine, trimethylamine, 2-ethylamino ethanol, histidine, procaine, and the like.

The composition may be administered in a manner compatible with the dosage formulation, and in such amount as will be prophylactically and/or therapeutically effective. The quantity to be administered depends on the subject to be treated, including, e.g., capacity of the subject's immune system to synthesize anti-inflammatory cytokines or to induce regulatory T cells, and the degree of protection desired. Suitable dosage ranges are of the order of several hundred micrograms active ingredient per vaccination with a preferred range from about 0.1 μg/g to 1000 μg/g, such as in the range from about 0.1 μg to 100 mg. Suitable regimens for initial administration and booster shots are also variable but are typified by an initial administration followed by subsequent inoculations or other administrations. Precise amounts of active ingredient required to be administered depend on the judgment of the practitioner and may be peculiar to each subject.

It will be apparent to those of skill in the art that the therapeutically effective amount of the FHA composition will depend, inter alia, upon the administration schedule, the unit dose of antigen administered, whether the FHA is administered in combination with other therapeutic agents, the immune status and health of the recipient, and the therapeutic activity of the particular FHA/antigen complex.

The composition may be given in a single dose schedule, or preferably in a multiple dose schedule. A multiple dose schedule is one in which a primary course of administration can include 1-10 separate doses, followed by other doses given at subsequent time intervals required to maintain and or reinforce the effect on the immune response, for example, at 1-4 months for a second dose, and if needed, a subsequent dose(s) after several months. Periodic administration at intervals of 1-5 years, usually 3 years, are desirable to maintain the desired levels of protection.

A series of vaccinations may be given, for example, at intervals of 3 months, or of four months, or of six months, between inoculations. Such a series may include, for example, 3 or 4 or 5 vaccinations in total. For vaccinations given to infants, a series of vaccination may be given, e.g., at birth or within the first week, and then at 6, 10 and 14 weeks of life. A series of vaccinations may be given at birth, and at 1, 3 and 6 months of life.

The composition may be administered for therapeutic use a number of times per week such as twice per week, weekly, a number of times per month, monthly for a number of weeks or months, for a year or for several years. The composition for therapeutic use may comprise the active ingredient on its own or in combination with a self-antigen. The therapy may also involve administration of other drugs either at the same time (either in the same formulation or separately) or at spaced time intervals.

A therapeutically effective dose may vary depending upon the route of administration and dosage form. Specific dosages may be adjusted depending on conditions of disease, the age, body weight, general health conditions, sex, diet of the subject, dose intervals, administration routes, excretion rate, and combinations of drugs. Any of the dosage forms containing effective amounts are well within the limits of routine experimentation. The compositions of the invention may also be administered in conjunction with other drugs including those used in the treatment of autoimmune disease. The compositions may also be administered alone using a similar dosage regime as used for other treatments of autoimmune disorders. The term “treatment” is intended to include an alleviation of symptoms associated with a disorder or disease, or the halt of further progression or worsening of those symptoms, or prevention or prophylaxis of the disease or disorder.

The course of the treatment can be followed by testing ex vivo cytokine production by cells of the immune system (recovered from blood samples) with and without in vitro stimulation with for example LPS. The assays can be performed using conventional reagents for culture of cells and quantification of cytokines using antibodies and the like. These techniques are commonly known to one skilled in the art.

The invention is not limited to the embodiments hereinbefore described which may be varied in detail.

### REFERENCES

1. McGuirk, P., and K. H. G. Mills. 2002. Pathogen-specific regulatory T cells provoke a shift in the Th1/Th2 paradigm in immunity to infectious diseases. Trends. Immunol. 23:450-455.
2. Lavelle, E., E. McNeela, M. E. Armstrong, O. Leavy, S. C. Higgins, and K. H. G. Mills. 2003. Cholera toxin promotes the induction of regulatory T cells as well as Th2 cells specific for bystander antigens by modulating dendritic cell activation. J. Immunol. 171:2384-2392.
3. McGuirk, P., C. McCann, and K. H. G. Mills. 2002. Pathogen-specific T regulatory 1 cells induced in the respiratory tract by a bacterial molecule that stimulates interleukin 10 production by dendritic cells: a novel strategy for evasion of protective T helper type 1 responses by Bordetella pertussis. J. Exp. Med. 195:221-231.
4. McGuirk, P. and K. H. G. Mills. 2000. Direct anti-inflammatory effect of a bacterial virulence factor: IL-10-dependent suppression of IL-12 production by filamentous hemagglutinin from Bordetella pertussis. Eur. J. Immunol. 30:415-422.
5. McGuirk, P., P. A. Johnson, E. J. Ryan, and K. H. G. Mills. 2000. Filamentous hemagglutinin and pertussis toxin from Bordetella pertussis modulate immune responses to unrelated antigens. J. Infect. Dis. 182, 1286-1289.
6. Ishibashi, Y., S. Claus, and D. A. Relman. 1994. Bordetella pertussis filamentous hemagglutinin interacts with a leukocyte signal transduction complex and stimulates bacterial adherence to monocyte CR3 (CD11b/CD18). J. Exp. Med. 180:1225-1233.
7. Asseman, C., Mauze, S., Leach, M. W., Coffman, R. L. and Powrie, F. 1999. An essential role for interleukin 10 in the function of regulatory T cells that inhibit intestinal inflammation. J. Exp. Med. 190:995-1004.
8. Sakaguchi, S., Takahashi, T., Yamazaki, S., Kuniyasu., Y., Itoh, M., Sakaguchi, N. and Shimizu, J. 2001. Immunologic self tolerance maintained by T-cell-mediated control of self-reactive T cells: implications for autoimmunity and tumor immunity. Microbes Infect. 3:911-8.

1-49. (canceled)

50. A pharmaceutical composition comprising substantially purified filamentous haemagglutinin (FHA) or derivative or mutant or fragment or variant or peptide thereof that is substantially endotoxin-free.

51. The pharmaceutical composition of claim 50, wherein said pharmaceutical composition is effective as an adjuvant for immunization with a self or foreign antigen.

52. The pharmaceutical composition of claim 50, further comprising antigen, wherein said antigen is selected from a self-antigen and a foreign antigen.

53. The pharmaceutical composition of claim 50 wherein said FHA comprises a product of cells activated by an FHA derivative or mutant or fragment or variant or peptide thereof.

54. The pharmaceutical composition of claim 51 wherein the self antigen is selected from any one or more of glutamic acid decarboxylase 65 (GAD 65), native DNA, a myelin protein, myelin proteolipid protein, acetylcholine receptor components, thyroglobulin, thyroid stimulating hormone (TSH) receptor, Japanese cedar pollen antigens, ragweed pollen antigens, rye grass pollen antigens, and dust mite antigens and feline antigens for animal, histocompatibility antigens, antigens involved in graft rejection, an altered peptide ligand, beta amyloid protein, amyloid precursor protein and collagen and peptides thereof.

55. The pharmaceutical composition of claim 54 wherein the antigens involved in graft rejection include antigenic components of the graft to be transplanted into the heart, lung, liver, pancreas, kidney for graft recipients and neural graft components.

56. (canceled)

57. The pharmaceutical composition as claimed in claim 54 wherein the myelin protein is selected from the group consisting of myelin basic protein, myelin oligodendrocyte glycoprotein synthetic peptide, a MOG peptide (35-55), and peptides thereof.

58-59. (canceled)

60. The pharmaceutical composition of claim 50, further comprising a TLR ligand.

61. The pharmaceutical composition of claim 60 wherein the TLR ligand is a pharmaceutically acceptable TLR ligand.

62. An immunomodulator comprising substantially purified FHA or derivative or mutant or fragment or variant or peptide thereof that is substantially endotoxin-free.

63. A recombinant FHA having immunomodulatory effects.

64. A vaccine comprising substantially purified FHA or derivative or mutant or fragment or variant or peptide thereof that is substantially endotoxin-free.

65. The vaccine of claim 64 further comprising an antigen.

66. The vaccine of claim 65 wherein the FHA and antigen are present in a ratio range of about 0.0:1 to about 100:1 by weight.

67. The vaccine of claim 65 wherein the FHA and antigen are present in a molar ratio of about 1:10 to about 10:1.

68. Antibodies to substantially purified FHA or derivative or mutant or fragment or variant or peptide thereof.

69. A method for preparing a substantially pure preparation of FHA comprising the steps of:
dialysing a preparation of FHA to denature the protein and expose contaminating endotoxin, and
removing residual contaminating endotoxin.

70. The method of claim 69 wherein the contaminating endotoxin is lipopolysaccharide (LPS).

71. The method of 69 wherein the endotoxin is removed using a detergent.

72. The method of claim 69 comprising the steps of:
priming a purification column;
adding the dialysed FHA preparation;
washing with detergent; and
eluting a substantially purified protein.

73. A method for the prophylaxis and/or treatment of an immune-mediated disorder comprising the step of administering an agent comprising filamentous haemagglutinin (FHA) or a derivative or mutant or fragment or variant or peptide thereof that is substantially endotoxin-free.

74. The method of claim 73, wherein said immune-mediated disorder is an autoimmune disease.

75. The method of claim 73 wherein the filamentous haemagglutinin (FHA) is derived from Bordetella pertussis or Bordetella bronchisepetica or Bordetella parapertussis or related molecules from other bacteria.

76. The method of claim 73 wherein the agent comprises a product of cells activated by FHA or derivative or mutant or fragment or variant or peptide thereof.

77. The method of claim 73 wherein the agent comprises FHA in combination with self or foreign antigens or peptides thereof.

78. The method of claim 73 wherein the agent has an activity selected from the group of activities consisting of: promotes the generation of Tr cells in response to a self antigen; acts as an immunomodulator in vivo to promote the induction of Tr cells to coadministered self or foreign antigens; modulates pro-inflammatory cytokine production; promotes the induction of anti-inflammatory cytokines: promotes IL-10 and TGF-β production by macrophages and dendritic cells: promotes IL-6 production by macrophages and dendritic cells: synergises with LPS to promote 1L-10, TGFβ and IL-6 production by macrophages and dendritic cells: induces expression of TGFβ mRNA; inhibits inflammatory cytokines, chemokines or other inflammatory mediators: promotes dendritic cell maturation into a semi-mature phenotype: promotes dendritic cell maturation following co-activation with TLR-ligands; inhibits TLR ligand-induced dendritic cell activation; and modulates inflammatory cytokine production induced by infection or trauma.

79. (canceled)

80. The method of claim 77 wherein the self antigen is selected from any one or more of glutamic acid decarboxylase 65 (GAD 65), native DNA, myelin basic protein, myelin proteolipid protein, acetylcholine receptor components, thyroglobulin, thyroid stimulating hormone (TSH) receptor, Japanese cedar pollen antigens, ragweed pollen antigens, rye grass pollen antigens, and dust mite antigens and feline antigens for animal, histocompatibility antigens, antigens involved in graft rejection and an altered peptide ligand.

81. The method of claim 80 wherein the antigens involved in graft rejection comprise antigenic components of the graft to be transplanted into the heart, lung, liver, pancreas, kidney of graft recipients and neural graft components.

82. The method of claim 77 wherein the self antigen is selected from any one or more of a myelin protein, beta amyloid protein, amyloid precursor protein and collagen and peptides thereof.

83. The method of claim 82 wherein the myelin protein is selected from the group consisting of myelin basic protein, myelin oligodendrocyte glycoprotein (MOG) synthetic peptide, a MOG peptide (35-55), and peptides thereof.

84-87. (canceled)

88. The method of claim 73 wherein the immunomodulatory effects of FHA on cells of the innate immune system are enhanced by co-activation with a Toll-like receptor ligand.

89. The method of claim 88 wherein the Toll-like receptor ligand is selected from the group of toll-like receptor ligands consisting of LPS, CpG motifs, dsRNA, Poly (I:C) and Pam3Cys.

90-94. (canceled)

95. The method of claim 78 wherein the inflammatory cytokine is selected from any one or more of TNF-α, IFN-γ, IL-2, IL-12, IL-1, IL-23 and IL-27.

96. The method of claim 78 wherein the inflammatory chemokine is macrophage inflammatory protein-1α or macrophage inflammatory protein 1β.

97-100. (canceled)

101. The method of claim 73 wherein FHA is in the form of an immmunomodulator, adjuvant, immunotherapeutic or anti-inflammatory agent.

102. (canceled)

103. The method of claim 73 wherein the immune-mediated disorder is selected from the group consisting of sepsis, acute inflammation induced by infection, acute inflammation induced by trauma, acute inflammation induced by injury, multiple sclerosis, Crohn's disease, inflammatory bowel disease, type 1 diabetes, rheumatoid arthritis, psoriasis, colitis, asthma, and atopic disease.

104-107. (canceled)

108. The method of claim 73 wherein the agent is in a form for oral, intranasal, intravenous, intradermal, subcutaneous or intramuscular administration.

109. The method of claim 108 comprising repeated administration of the agent.

110. The method claim 73 wherein the immune-mediated disorder is selected from the group consisting of diabetes mellitus, arthritis, rheumatoid arthritis, juvenile rheumatoid arthritis, osteoarthritis, psoriatic arthritis, myasthenia gravis, systemic lupus erythematosis, autoimmune thyroiditis, dermatitis, atopic dermatitis, eczematous dermatitis, Sjogren's Syndrome, keratoconjunctivitis sicca secondary to Sjogren's Syndrome, alopecia areata, allergic responses due to arthropod bite reactions, aphthous ulcer, iritis, conjunctivitis, keratoconjunctivitis, ulcerative colitis, asthma, allergic asthma, cutaneous lupus erythematosus, scieroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, interstitial lung fibrosis, Alzheimer's disease and coeliac disease.
